(19) United States
(12) Patent Application Publication
(10) Pub. No.: US 2020/0383632 A1
(43) Pub. Date: Dec. 10, 2020

(54) MR-Compatible Device For Non-Invasive Assessment of Muscle Compliance

(71) Applicant: The Trustees of the University of Pennsylvania, Philadelphia, PA (US)

(72) Inventors: Anil Chauhan, Drexel Hill, PA (US); Lawrence Dougherty, Wenonah, NJ (US); James J Pilla, Kennett Square, PA (US)

(21) Appl. No.: 16/888,195

(22) Filed: May 29, 2020

Related U.S. Application Data
(60) Provisional application No. 62/855,042, filed on May 31, 2019.

(51) Int. Cl. A61B 5/00; A61B 5/055; G01R 33/34

(52) U.S. Cl. CPC A61B 5/4519; G01R 33/34; A61B 5/055; A61B 5/0053

(57) ABSTRACT

Provided are devices and methods that deform tissue synchronous with an MR imaging cycle, as well as devices and methods for determination of dynamic determination of one or more mechanical properties of the tissue, including properties of muscle tissue. Such determination can be performed with and without imaging of the tissue being evaluated.

### RELATED APPLICATIONS

The present application claims priority to and the benefit of U.S. patent application No. 62/855,042, “An MR-Compatible Device For Non-Invasive Assessment Of Abdominal Muscle Compliance” (filed May 31, 2019), the entirety of which application is incorporated herein by reference for any and all purposes.

### TECHNICAL FIELD

The present disclosure relates to the field of magnetic resonance imaging and to the field of tissue deformation devices.

### BACKGROUND

Two million abdominal surgeries are performed annually in the United States, and ventral hernia (VH) is among the most common and serious complications. The incidence of VH is approximately 13%, but as high as 70% in high-risk populations. Failed repairs generate un-closable midline defects that require mesh bridging—native fascia cannot be closed and the mesh is ‘bridged’ across the fascia.

Bridging is a relevant and clinically meaningful outcome, since repairs fail in 44 to 100% of cases and significant costs are incurred. The current state of hernia repair relies heavily on clinical evaluation of patients, which is ultimately a poor predictor of outcomes for patients going into surgery. There are currently no reliable data, standard imaging modalities, or guidelines available to predict successful fascial closure in hernia repair or to predict successful approaches in other surgeries. Accordingly, there is a long-felt need in the art for methods and devices that allow for determination and/or prediction of the mechanical properties of tissue, whether that tissue be abdominal tissue or other tissue.

### SUMMARY

In meeting the long-felt needs described above, the present disclosure provides, inter alia, methods that allow non-invasive assessment of muscle compliance using a device to deform the abdominal muscle combined with MR tissue tagging.

In one aspect, the present disclosure provides methods, comprising: with a force applicator, deforming a region of a tissue synchronous with at least one magnetic resonance (MR) imaging sequence that comprises MR tissue tagging.

In another aspect, the present disclosure provides methods, comprising: deforming a region of a tissue and while the tissue is at a deformed state and with at least one MR imaging sequence that comprises MR tissue tagging, monitoring displacement at a plurality of time points so as to estimate at least one characteristic of the tissue. Monitoring can also be accomplished by monitoring the tissue at the start of (or before) the deforming and at the completion of the deformation.

Also provided are systems for magnetic resonance (MR) imaging of an object, comprising: a force applicator configured to effect exertion of a force on the object so as to deform the object; the system further comprising a radio frequency (RF) coil, the RF coil being optionally configured as receive only or as a transmit and receive coil, the RF coil being optionally being configured as a single loop or as a multi-coil array, the force applicator optionally being configured to effect exertion of the force at least partially synchronous with at least one magnetic resonance (MR) imaging sequence (such as one that comprises MR tissue tagging), the system further comprising a signal source configured to give rise to a signal that allows for measurement of motion of the force applicator; the system optionally comprising an MR imaging sequence using tissue tagging to measure 2D and/or 3D tissue displacements.

Further provided are systems, comprising: a force applicator configured to apply a compressive force to a subject's tissue synchronous with a cycle during MR sequence acquisition, a radio frequency (RF) coil; the RF coil being configured as a receive only or a transmit and receive coil, the RF coil being configured as a single loop or a multi-coil array, and an MR imaging sequence to generate images synchronous with tissue displacement.

Additionally provided are methods, comprising: synchronous with at least one magnetic resonance (MR) sequence that comprises MR tissue tagging, applying a force to a location on a subject so as to displace adipose tissue of the subject; collecting at least one signal related to the application of the force; and relating at least one signal to an estimated mechanical property of the adipose tissue.

Further provided are systems, the systems comprising: a MR-compatible force applicator configured to apply a force to a location on a subject synchronous with at least one magnetic resonance (MR) sequence that comprises MR tissue tagging, the force being applied so as to displace adipose tissue of the subject; and a processor, the processor configured to relate a signal related to the application of the force to an estimated mechanical property of the adipose tissue.

Also disclosed are methods, comprising: relating a displacement velocity, a bending modulus, or an elastic modulus of the tissue to a physiological condition of the tissue following application of a force to a region of a subject.

Further provided are methods, comprising: with a force applicator that travels a distance, applying a force to a subject (e.g., to their abdomen); relating one or both of the force and the distance to an estimated mechanical property of a muscle tissue of the subject; the relating being at least partially based on an estimated mechanical property of an adipose tissue.

Additionally provided are methods, comprising: with a force applicator that travels a distance, applying a force to a subject (e.g., to their abdomen); relating one or both of the force and the distance to an estimated mechanical property of a muscle tissue of the subject, the relating being accomplished without imaging the muscle tissue.

Also provided are systems, comprising: a force applicator configured to apply a force to a location on a subject, the force being applied so as to displace adipose tissue of the subject; and a processor, the processor configured to relate a signal related to the application of the force to an estimated mechanical property of the adipose tissue.

Further provided are systems, comprising: a force applicator configured to apply a force to a location on a subject, the force being applied so as to displace adipose tissue of the subject; and a processor, the processor configured to relate a signal related to the application of the force to an estimated mechanical property of muscle tissue adjacent to the adipose tissue.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIG. 1 provides an exploded view of an exemplary device according to the present disclosure;

FIG. 2 provides MR tagged images acquired during abdominal displacement using a device according to the present disclosure;

FIG. 3 provides tissue displacement shown as color overlay on abdominal muscle with usage of a device according to the present disclosure;

FIG. 4 provides maximum principal strain (stretch) shown as vectors in the region of abdominal compression;

FIG. 5 provides maximum principal strain shown as (color) overlay on abdominal muscle with usage of a device according to the present disclosure;

FIG. 6A provides a bottom view of an exemplary device (with plunger inserted);

FIG. 6B provides an alternative view of an exemplary device;

FIG. 6C provides a side view of the device of FIG. 6;

FIG. 6D provides an alternative side view of the device of FIG. 6C (with plunger removed);

FIG. 7A provides a top view of an alternative device according to the present disclosure;

FIG. 7B provides a bottom view of the device of FIG. 7A;

FIG. 7C provides an alternative side view of the device of FIG. 7B (with plunger removed);

FIG. 7D provides a top view of the device of FIG. 7C;

FIG. 8A provides a tagged MR image from a normal subject;

FIG. 8B provides a strain map of the MR image of FIG. 8A;

FIG. 8C provides a strain profile for the tissue imaged in FIGS. 8A and 8B;

FIG. 9A provides a tagged MR image from a hernia subject;

FIG. 9B provides a strain map of the MR image of FIG. 9A;

FIG. 9C provides a strain profile for the tissue imaged in FIGS. 9A and 9B;

FIG. 10 provides time resolved surface renderings of 3D MR tissue-tagged acquisitions of the abdominal wall;

FIG. 11 provides tissue flow profiles of abdominal muscle undergoing deformation, demonstrating (by the width of each plot) the elasticity of the muscle varying with the profile angle;

FIGS. 12A-12B provide illustrative temporal response to deformation at three locations across the abdominal muscle for a normal (FIG. 12A) and abnormal/hernia (FIG. 12B) subject. Without being bound to any particular theory, the time delay between application of the deforming force and subsequent displacement of the lateral positions is an indicator of muscle stiffness. Hernia subjects (FIG. 12B) showed a 25 ms-50 ms greater propagation delay in tissue displacement and a 26% increase in tissue velocity (at 2 cm) as compared to normal subjects (FIG. 12A).

FIGS. 13A-13B illustrate the effect of abdominal adipose tissue thickness layer on muscle displacement using hernia device and MR imaging with adipose tissue thickness of 25.36 mm (FIG. 13A) and of 8.19 mm (FIG. 13B). Graphs show the displacement of the adipose tissue and abdominal muscle for difference adipose tissue layers. The thicker the adipose layer the less muscle displacement is observed. Information concerning the thickness and properties of the adipose tissue is imperative for an accurate measure and abdominal muscle stiffness and how it correlates with hernia formation.

FIG. 14 provides compressive properties of abdominal wall adipose tissue measured using device and MR imaging. For thicker adipose layer the ratio of compression is greater compared to narrower layer. Both layers reach an asymptote at different thicknesses indicating that the applied force is transferred to the muscle at different levels depending on adipose layer thickness. The point at which total applied force is transferred to the muscle layer is important for determining abdominal properties.

FIGS. 15A and 15B provide example results from FEA modeling showing the effect of adipose tissue thickness on muscle displacement. The simulation correlates well with the observed hernia device and MR imaging results.

FIG. 16 provides applied force versus adipose tissue displacement generated from FEA. Adipose tissue thickness significantly alters the measured force for the same displacement.

FIG. 17 provides applied force versus muscle tissue displacement generated from FEA. Adipose tissue thickness significantly alters the measured force and displacement of the underlying muscle layer.

FIG. 18 provides an example FEA model of the abdominal wall and adipose tissue layer. An abdominal wall is modeled as beam supported on two opposing sides with a concentrated force applied at center; a model with adipose tissue of 5 mm is on the left and a model with adipose tissue of 20 mm is on the right. This configuration is analogous to the flexural testing method to measure the stiffness of material under a concentrated load. The adipose tissue is modeled as a material loosely connected to the underlying muscle.

FIG. 19 provides an illustration of a finite element model of abdominal wall and device. The abdominal wall is modeled as a supported beam with two opposite sides fixed. Force is applied at center of beam using an object with a semi-spherical contact point to represent device. It should be understood that although the abdominal wall is modeled as a beam, other musculature (i.e., besides the abdominal wall) can be modeled as a beam.

FIG. 20 provides an exemplary view of abdominal wall and device model displacement, highlighting abdominal wall displacement in the z-direction for an applied device force.

FIG. 21 provides a graph of abdominal wall and device model z-displacement versus elastic modulus.

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints, 2 grams and 10 grams, and all the intermediate values). The endpoints of the ranges and any values disclosed herein are not limited to the precise range or value; they are sufficiently imprecise to include values approximating these ranges and/or values.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. In at least some instances, the approximating language may correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4. Further, the term “comprising” should be understood as having its open-ended meaning of “including,” but the term also includes the closed meaning of the term “consisting.” For example, a composition that comprises components A and B may be a composition that includes A, B, and other components, but may also be a composition made of A and B only. Any documents cited herein are incorporated by reference in their entireties for any and all purposes.

Certain aspects of the disclosed technology are described in relation to or with respect to a patient's abdomen. It should be understood, however, that the disclosed technology is not limited to uses related to the abdomen, and that the disclosed technology can be used to determine mechanical properties of musculature besides that in the abdomen, e.g., thighs, extremity musculature, and paraspinal musculature, to provide but a few examples.

### FIGURES

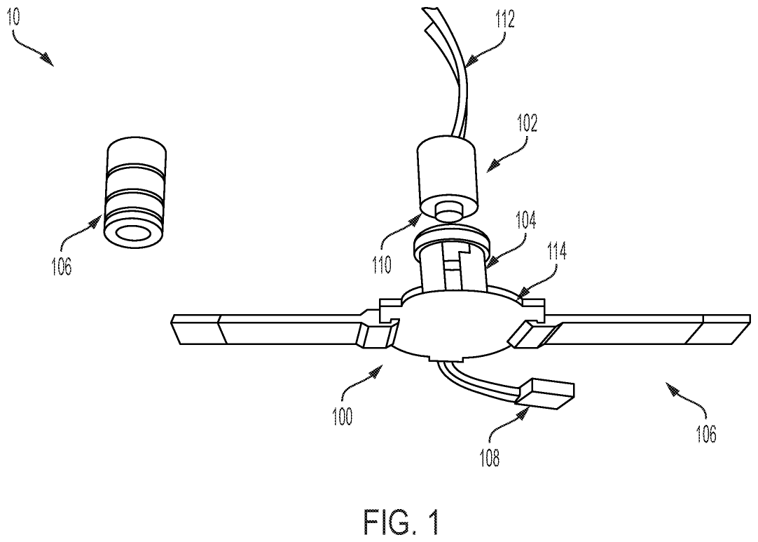

FIG. 1 provides a depiction of an illustrative device 10. As shown, device 10 can include a base 114. Device 10 can also include a holder portion 104, which holder portion can be configured to receive air cylinder 112. Air cylinder 112 can comprise a piston (not shown in its entirety), which piston can include an end 110 that extends through an opening (not labeled) in base 114. End 110 can be rounded, but can also be tapered, flared, or even flat. Device 10 can also include connection 112, which connection can allow for control of air cylinder 112 and the extension of the piston. One or more spacers 106 can be placed into holder portion 104 so as to space the end 110 from the bottom of base 114. By spacing end 110 at a distance from the bottom of base 114, a user can control the distance that end 110 extends beyond base 114.

A force applicator can be configured to provide force application in a continuous manner, e.g., via extension of the end of a piston. The end of the piston can extend continuously. This can be done by extension at a constant rate, but can also be by extension by non-constant rate, or even in a step-wise or interval manner. In this way, the disclosed technology can deform tissue in a constant manner. The disclosed technology can also deform tissue in a non-constant manner. The disclosed technology can also deform tissue in a step-wise manner, e.g., by extending a piston end by 1 cm, maintaining the piston rod in that position, then extending the end by another 1 cm, maintaining the piston rod in that position, and by repeating the foregoing steps. Tissue can be deformed during at least some of a magnetic resonance (MR) imaging sequence (such as one that comprises MR tissue tagging). Tissue can be deformed in a cyclic manner, e.g., by repeating one or more deformation-causing motions. Deformation can be accomplished by application of two or more different deformation-causing motions, e.g., an advancement of a piston end of 1 cm in 1 second followed by advancement of the piston end of 2 cm in 1 second.

Device 10 can also include an MR receiver coil 110. A device can also include a MR transmission coil (not shown). A device can also include a hold down strap 106; a device can also include other components (e.g., an elastic band) that is used to maintain the device in position on a patient. Device 10 can further include connector 108, which connector can be used to send/receive information from the device and also provide the user with control over the device.

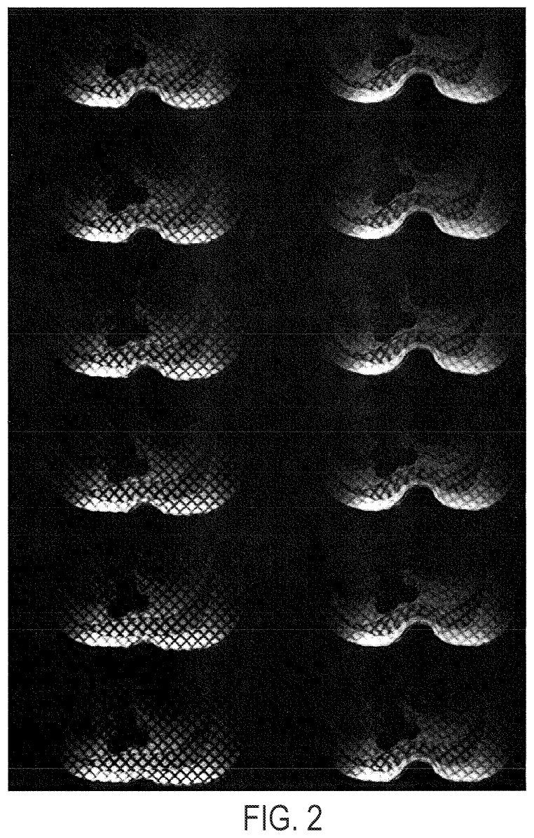

FIG. 2 provides a sequence of images showing abdominal tissue deformation (left to right, top row to bottom row) resulting from use of a device according to the present disclosure.

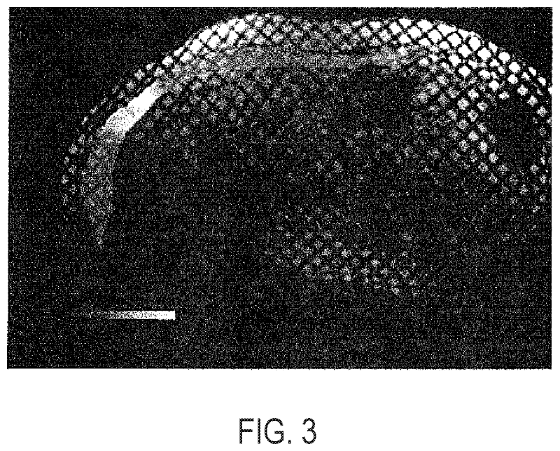

FIG. 3 provides tissue displacement shown as color overlay on abdominal muscle with usage of a device according to the present disclosure.

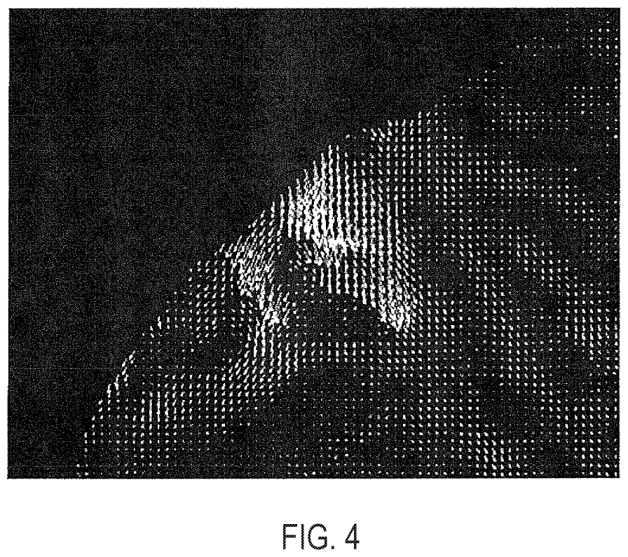

FIG. 4 provides maximum principal strain (stretch) shown as vectors in the region of abdominal compression.

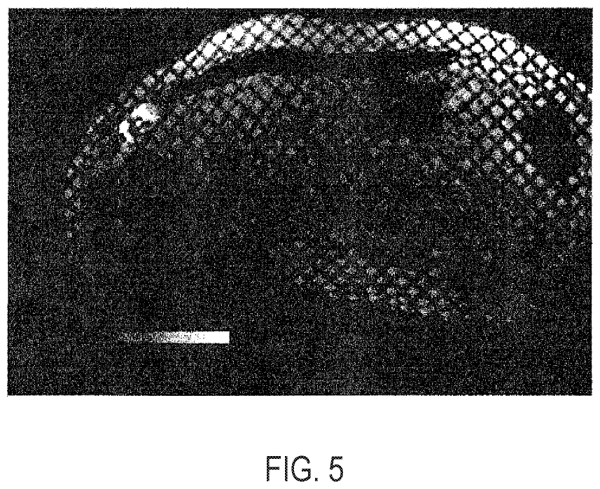

FIG. 5 provides maximum principal strain shown as color overlay on abdominal muscle with usage of a device according to the present disclosure.

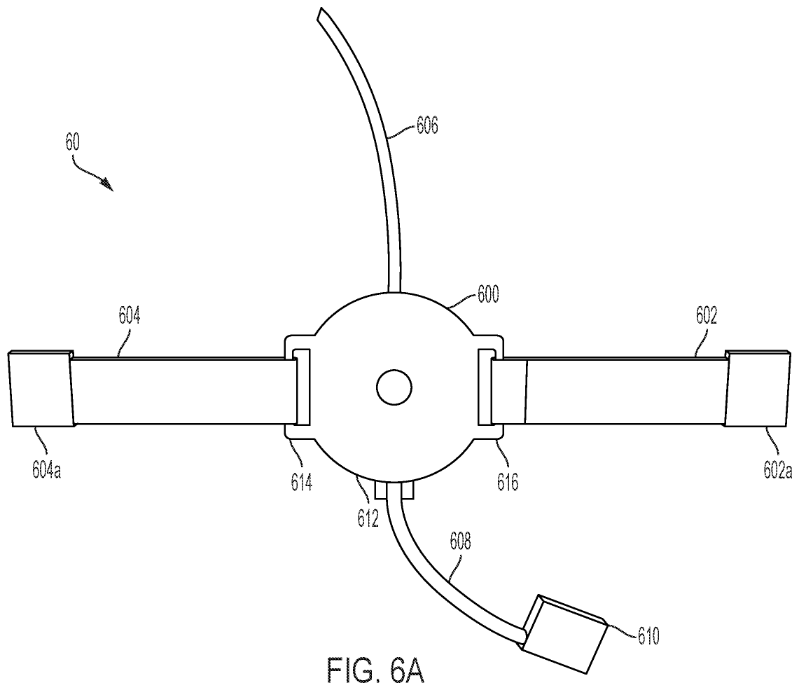

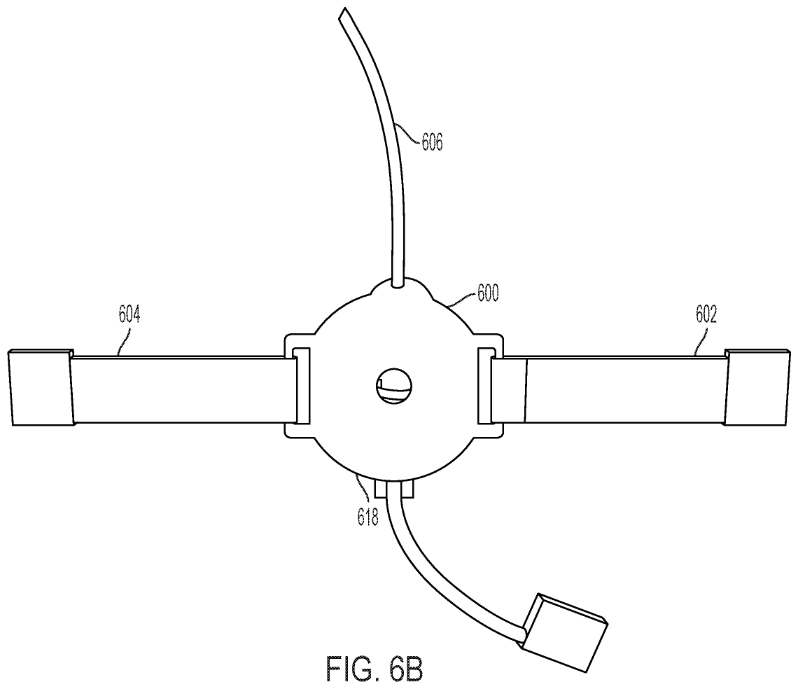

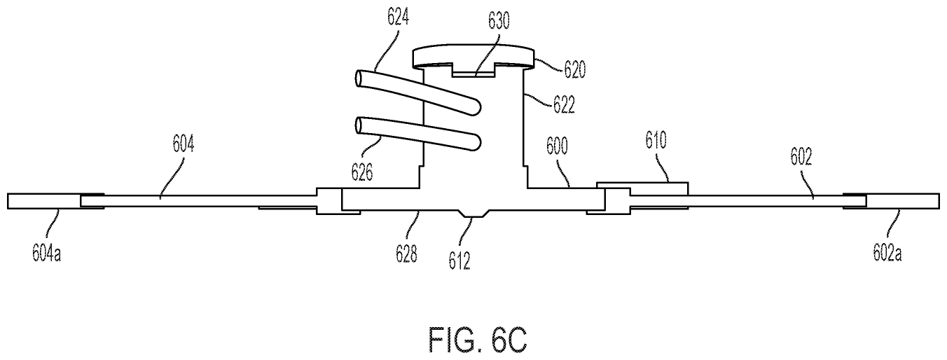

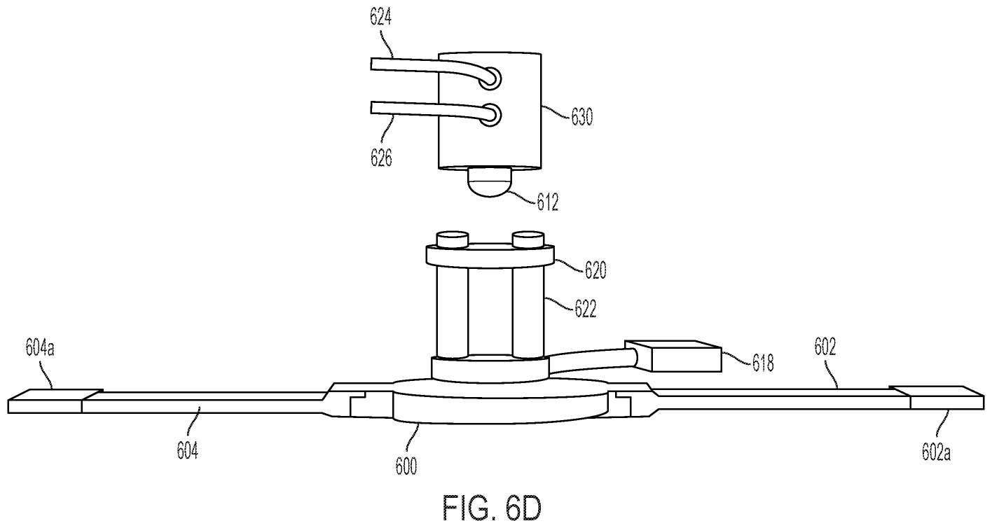

FIG. 6A provides a bottom view of a device 60. Device 60 can include a base 600, which base can support the various other components of device 60. The device can include connection 606, which connection can be used to send and/or receive data from the device. The device can also include connection 608 with interface module 610, which can also be used to send and/or receive data from the device.

The device can include strap 602 (with end 602a) and/or strap 604 (with end 604a). The straps and their ends can be used to secure the device into a location, e.g., via straps that connect to one another around the patient, via straps that connect to a platform or support, or in another manner. The device can include bracket 606 and/or bracket 614 that engage with straps 602 and 604. Also shown is the end 612 of a piston (not labeled) of the device that advances toward a patient's tissue so as to deform the tissue. End 612 can be rounded, but can also be flared, tapered, or even flat.

FIG. 6B provides an alternative bottom view of device 60 from FIG. 6A, with piston (not labeled) removed so as to show opening 618 of base 600. As shown, the end of the piston (not labeled) can extend through opening 618.

FIG. 6C provides a side view of device 60. As shown, the device can include force applicator 630. The force applicator can include connection 624 and/or connection 626, which can allow for transmission of data and/or signals to/from the force applicator or otherwise provide the user with control over the force applicator. The force applicator can comprise a piston having end 612. The device can include a holder portion 622 that is configured to receive the force applicator 630. The holder portion can include a collar 620. The device can also include RF coil 628, which coil can be a transmission coil, a receiving coil, or both. Coil 628 can be integrated into the base 600, but can also be removable.

FIG. 6D provides an alternative side view of the device of FIG. 6C, showing the force applicator 630 removed from holder portion 622. It should be understood that a force applicator can be electrically actuated, but can also be hydraulic or pneumatically actuated. In some embodiments, fluid (e.g., air, hydraulic fluid) can be communicated to the force actuator to effect force application by the force actuator.

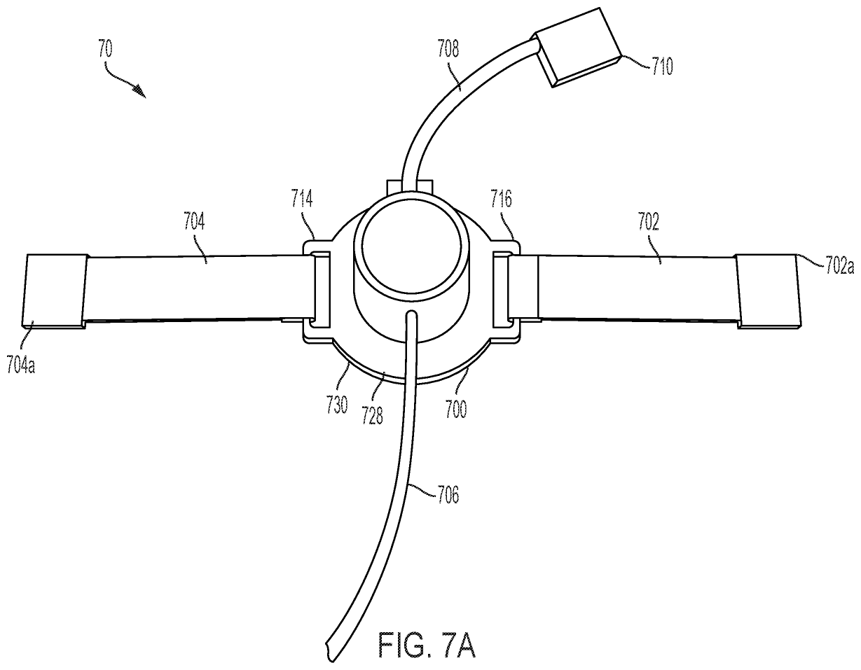

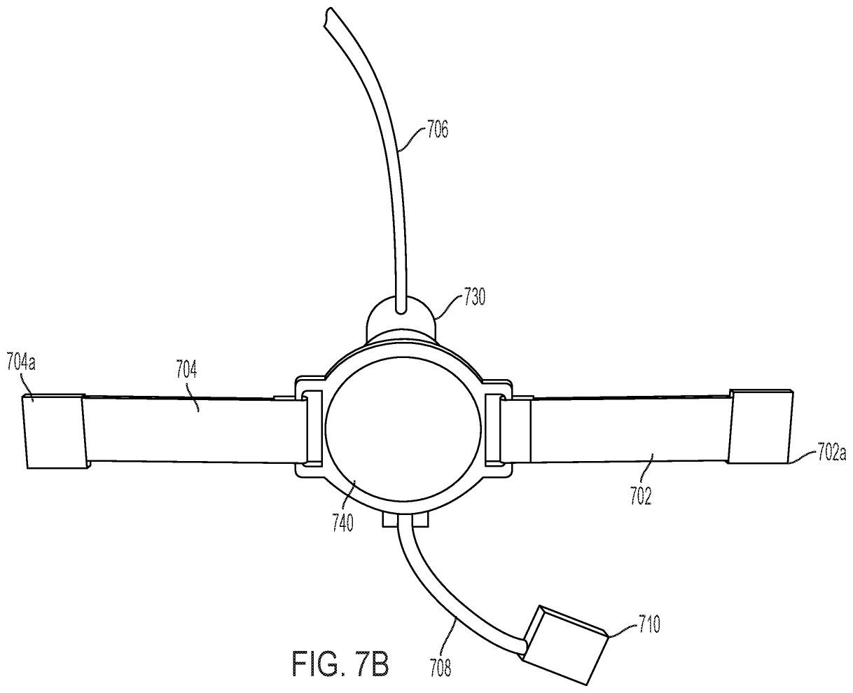

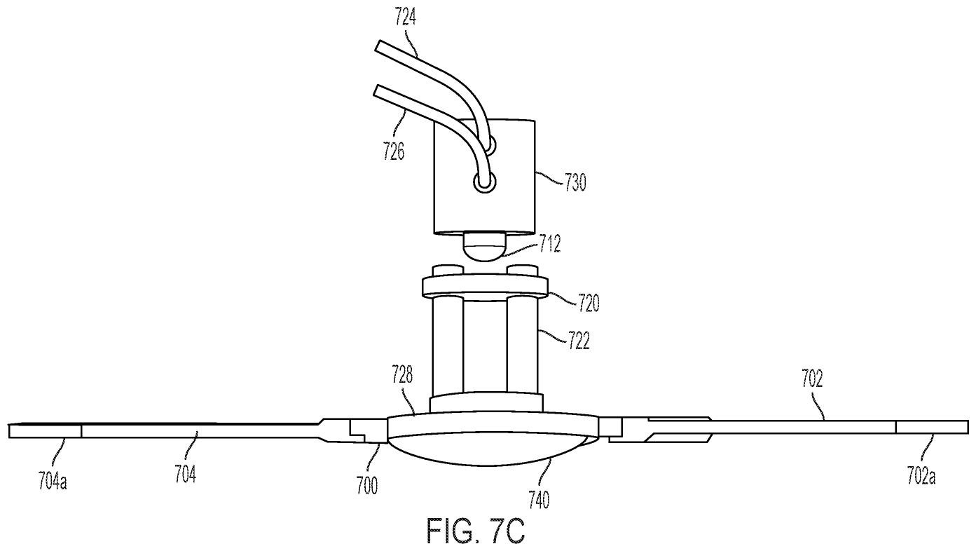

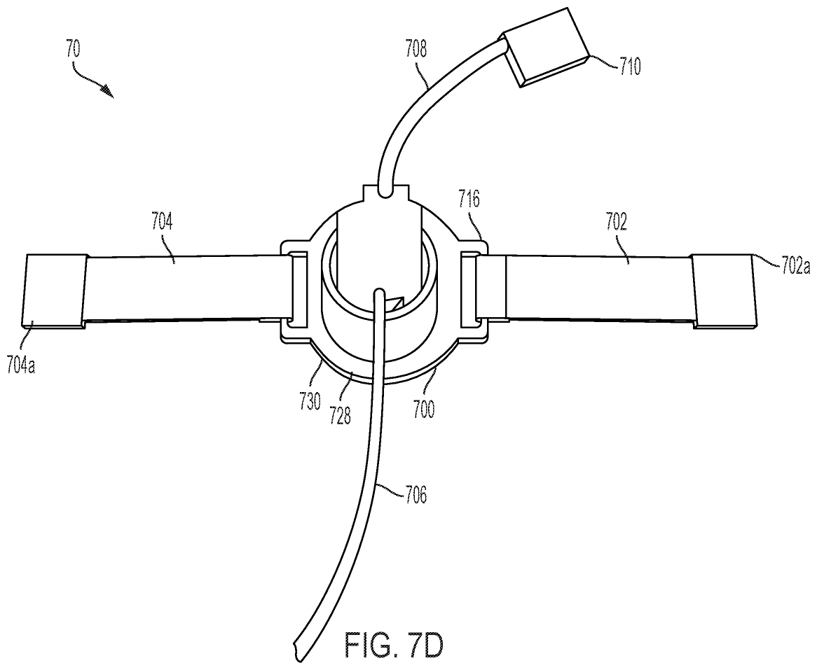

FIG. 7A provides a top view of an alternative device 70 according to the present disclosure. Device 70 can include a base 700, which base can support the various other components of device 70. The device can include connection 706, which connection can be used to send and/or receive data and/or signals from the device. The device can also include connection 708 with interface module 710, which can also be used to send and/or receive data from the device.

The device can include strap 702 (with end 702a) and/or strap 704 (with end 704a). The straps and their ends can be used to secure the device into a location, e.g., via straps that connect to one another around the patient, via straps that connect to a platform or support, or in another manner. The device can include bracket 706 and/or bracket 714 that engage with straps 702 and 704. Also shown is the end 712 of a piston (not labeled) of the device that advances toward a patient's tissue so as to deform the tissue. End 712 can be rounded, but can also be flared, tapered, or even flat. Also shown is force applicator 730. The force applicator can be held in place by a holder portion (not labeled). As shown, a device can include an RF coil 728. The coil can be a transmission coil, a receiving coil, or both. Coil 728 can be integrated into base 700, but can also be removable.

FIG. 7B provides a bottom view of the device 70 of FIG. 7B. As shown, the device can include membrane 740. The membrane can be flexible, but can also be rigid (e.g., dome-shaped) in some embodiments.

FIG. 7C provides an alternative side view of the device of FIG. 7B, showing the force applicator 730 removed from holder portion 722. It should be understood that a force applicator can be electrically actuated, but can also be hydraulic or pneumatically actuated. In some embodiments, fluid (e.g., air, hydraulic fluid) can be communicated to the force actuator to effect force application by the force actuator.

As shown, force applicator 730 can include connection 724 and/or connection 726, which can allow for transmission of data and/or signals to/from the force applicator or otherwise provide the user with control over the force applicator. The force applicator can comprise a piston having end 712. The device can include a holder portion 722 that is configured to receive the force applicator 730. The holder portion can include a collar 720.

As shown, a device can include an RF coil 728. The coil can be a transmission coil, a receiving coil, or both. Coil 728 can be integrated into base 700, but can also be removable.

FIG. 7D provides a top view of alternative device 70 in FIG. 7A, according to the present disclosure. Device 70 can include a base 700, which base can support the various other components of device 70. The device can include connection 706, which connection can be used to send and/or receive data and/or signals from the device. The device can also include connection 708 with interface module 710, which can also be used to send and/or receive data from the device.

The device can include strap 702 (with end 702a) and/or strap 704 (with end 704a). The straps and their ends can be used to secure the device into a location, e.g., via straps that connect to one another around the patient, via straps that connect to a platform or support, or in another manner. The device can include bracket 706 and/or bracket 714 that engage with straps 702 and 704. Also shown is the end 712 of a piston (not labeled) of the device that advances toward a patient's tissue so as to deform the tissue. End 712 can be rounded, but can also be flared, tapered, or even flat. Also shown is force applicator 730. The force applicator can be held in place by a holder portion (not labeled). As shown, a device can include an RF coil 728. The coil can be a transmission coil, a receiving coil, or both. Coil 728 can be integrated into base 700, but can also be removable.

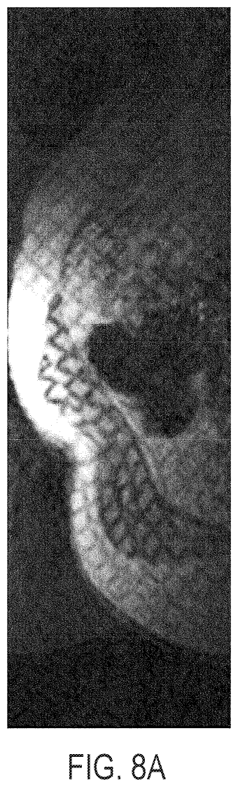

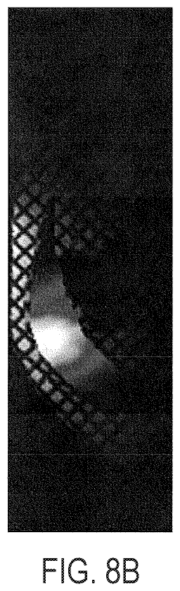

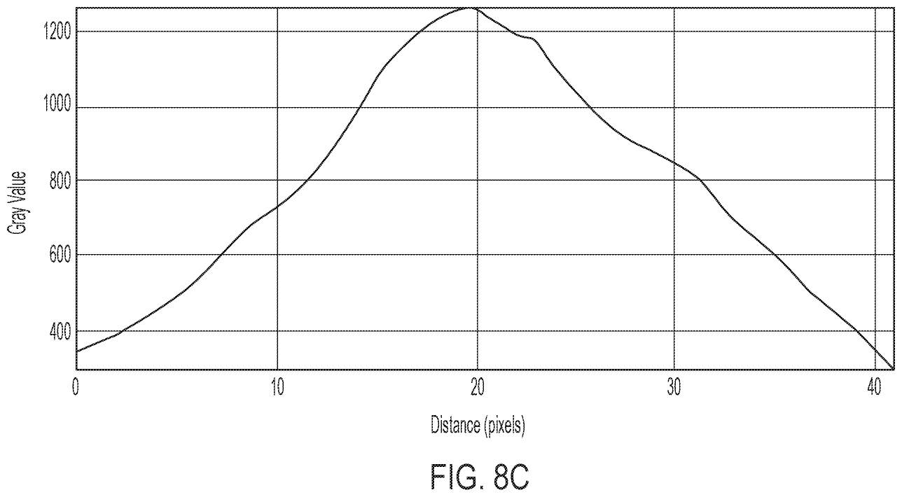

FIG. 8A provides a tagged MR image from a normal subject with the MR image being taken of the normal subject's tissue (external oblique, internal oblique, and transversus Abdominus) as the tissue is subjected to a deformation applied by the disclosed technology.

FIG. 8B provides a strain map of the tissue shown in FIG. 8A. FIG. 8C provides a strain profile (given by Gray Value as a function of distance, in pixels) for the tissue shown in FIG. 8B. As shown, the normal subject's tissue exhibits a relatively high compliance.

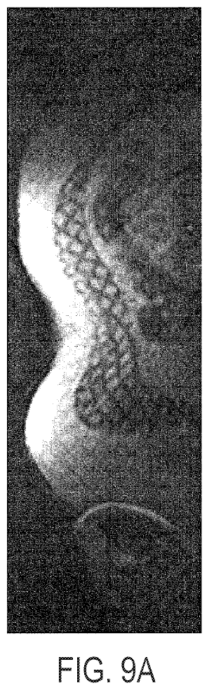

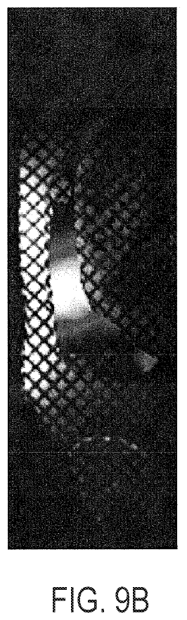

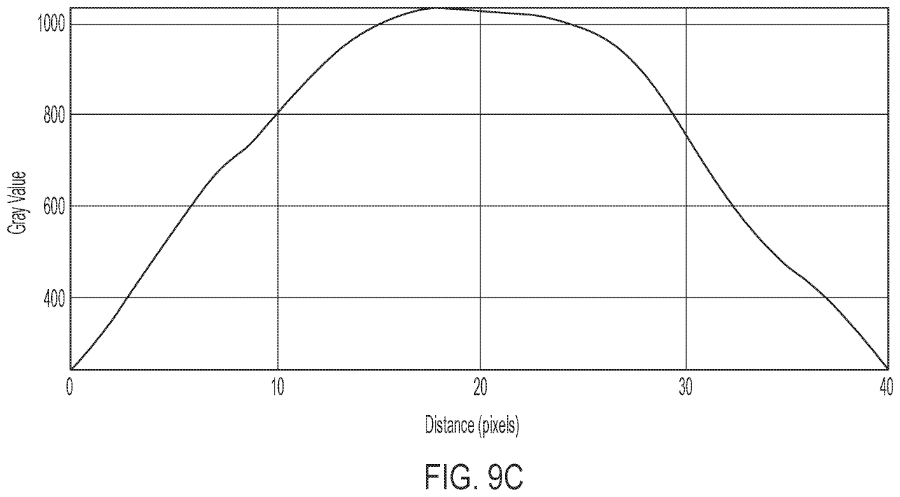

As a comparison to FIGS. 8A-8C, FIG. 9A provides a tagged MR image from a hernia subject; the MR image is taken of the normal subject's tissue as the tissue is subjected to deformation by the disclosed technology. FIG. 9B provides a strain map of the tissue shown in FIG. 9A. FIG. 9C provides a strain profile (again given by Gray Value as a function of distance, in pixels) for the tissue shown in FIG. 9B. As shown, the hernia subject's tissue exhibits a relatively low compliance (in terms of strain), as compared to the tissue of the normal subject.

For the results provided in FIGS. 8A-8C and 9C-9C, the force applied was from 13-44 Newtons. Force was applied cyclically on/off, with a period of 1 second.

To acquire an image, ˜20 cycles were used. Multiple images were acquired at different slice orientation angles. Total imaging time was <30 minutes, including set-up and scouts. Scans were acquired on a Siemens Magnetom MR scanner (Trio™ model; 3 Tesla) running VB17. The pulse sequence used was 2D SPGR gated multiphase acquisition with tissue tagging.

Deformation was performed using an external device (according to the present disclosure) that included an MR compatible pneumatically actuated air cylinder (IPS, Irvine, Calif.) that was mounted to a custom-made RF coil.

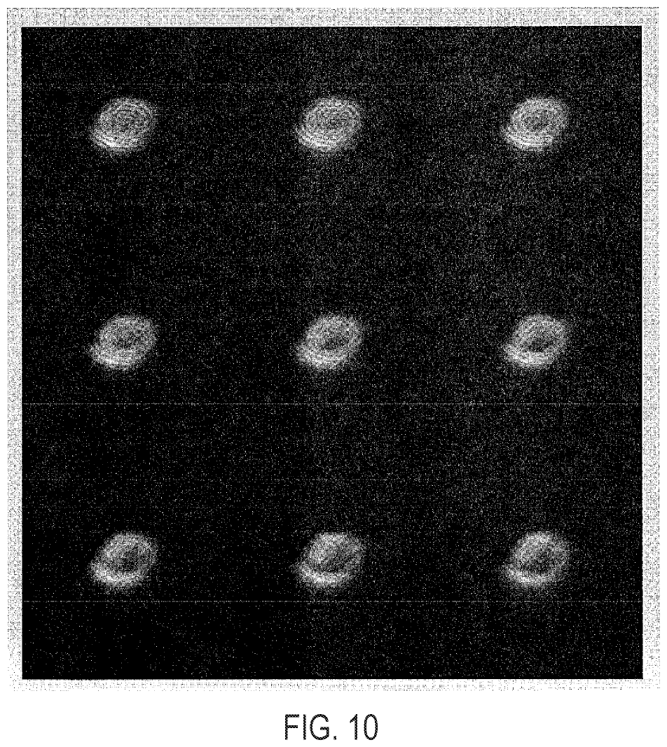

FIG. 10 provides time resolved surface renderings of 3D MR tissue-tagged acquisitions of the abdominal wall for an exemplary subject. Two-dimensional images were sequentially acquired in a radial pattern during subject breath-holds and interpolated onto a rectangular 3D grid. The subject had a previous hernia repair. As shown, the disclosed methods allow for sensitivity to muscle fiber angle and can improve tissue flow and strain estimation by considering 3D motion.

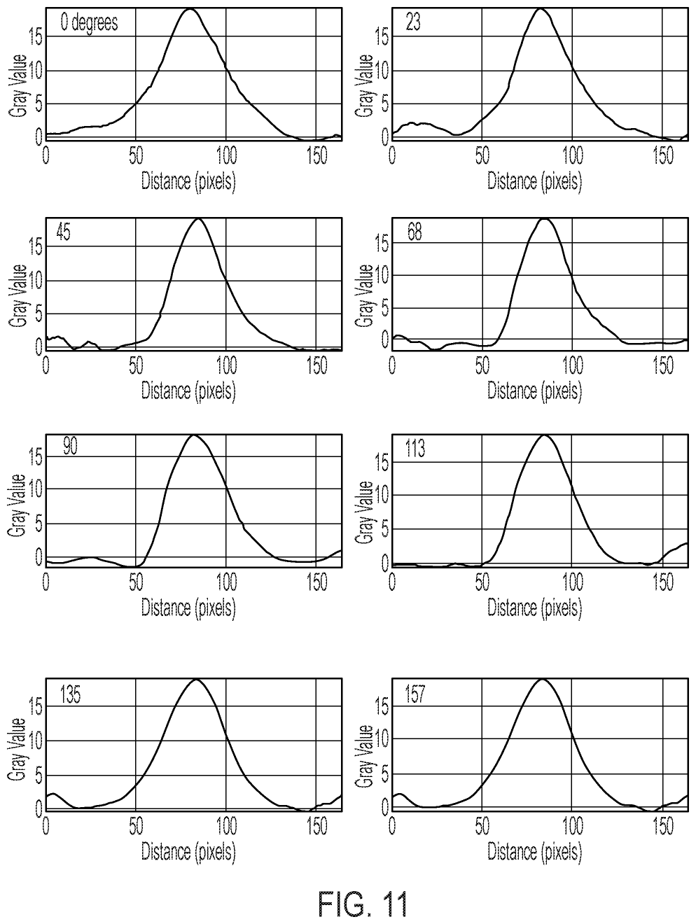

FIG. 11 provides tissue flow profiles of abdominal muscle of an exemplary subject. As shown, these profiles demonstrate (by the width of each plot) the elasticity of the muscle varying with the profile angle.

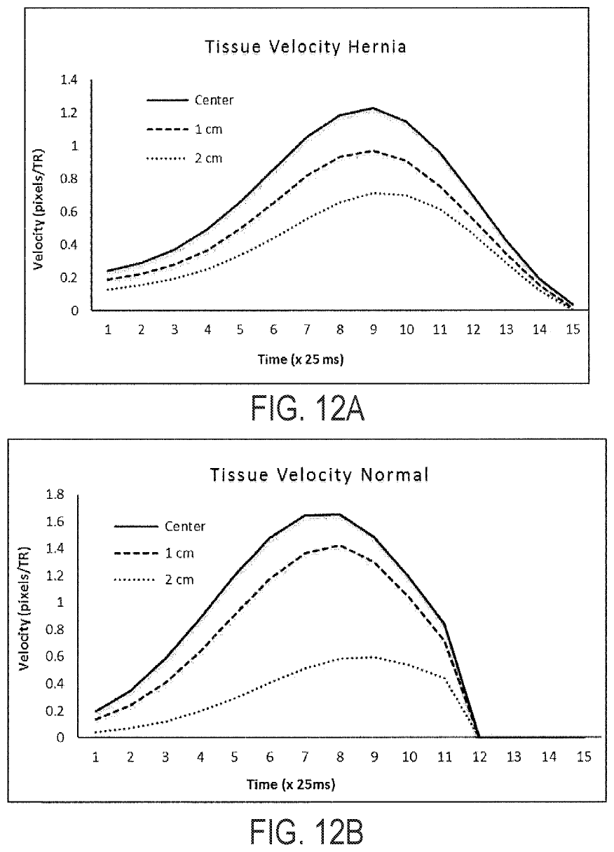

As shown in FIGS. 12A-12B, tissue velocity and the transverse wave speed can be significant measures of muscle stiffness. In these FIGs. propagation of deformation was calculated by comparing the time resolved tissue velocities at 3 points in the muscle. ROI's are placed in the abdominal muscle at the center axis of applied force, 1 cm and 2 cm laterally. The measured velocities are temporally interpolated using a 6th-order polynomial fit from which peak velocities and time of peak are obtained. The difference between the time to peak velocity from each lateral point and the center, normalized by the ratio of the peak velocities, are calculated.

FIGS. 12A-12B provide illustrative temporal response to deformation at three locations across the abdominal muscle for a normal (FIG. 12A) and abnormal/hernia (FIG. 12B) subject. Without being bound to any particular theory, the time delay between application of the deforming force and subsequent displacement of the lateral positions is an indicator of muscle stiffness. Hernia subjects (FIG. 12B) showed a 25 ms-50 ms greater propagation delay in tissue displacement and a 26% increase in tissue velocity (at 2 cm) as compared to normal subjects (FIG. 12A).

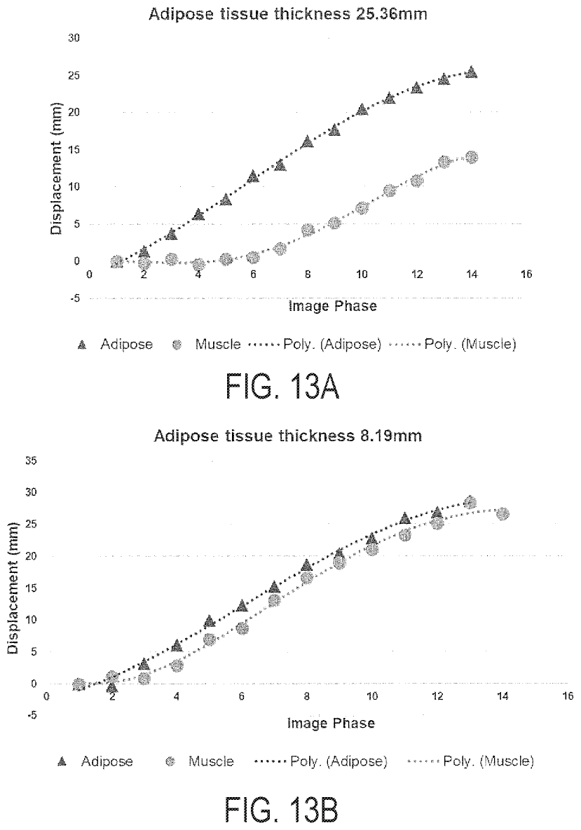

FIGS. 13A-13B illustrate the effect of abdominal adipose tissue thickness layer on muscle displacement using hernia device and MR imaging with adipose tissue thickness of 25.36 mm (FIG. 13A) and of 8.19 mm (FIG. 13B). These graphs show the displacement of the adipose tissue and abdominal muscle for different adipose tissue layers; the thicker the adipose layer the less muscle displacement is observed. Information concerning the thickness and properties of the adipose tissue is thus useful for an accurate measure and abdominal muscle stiffness and how it correlates with hernia formation.

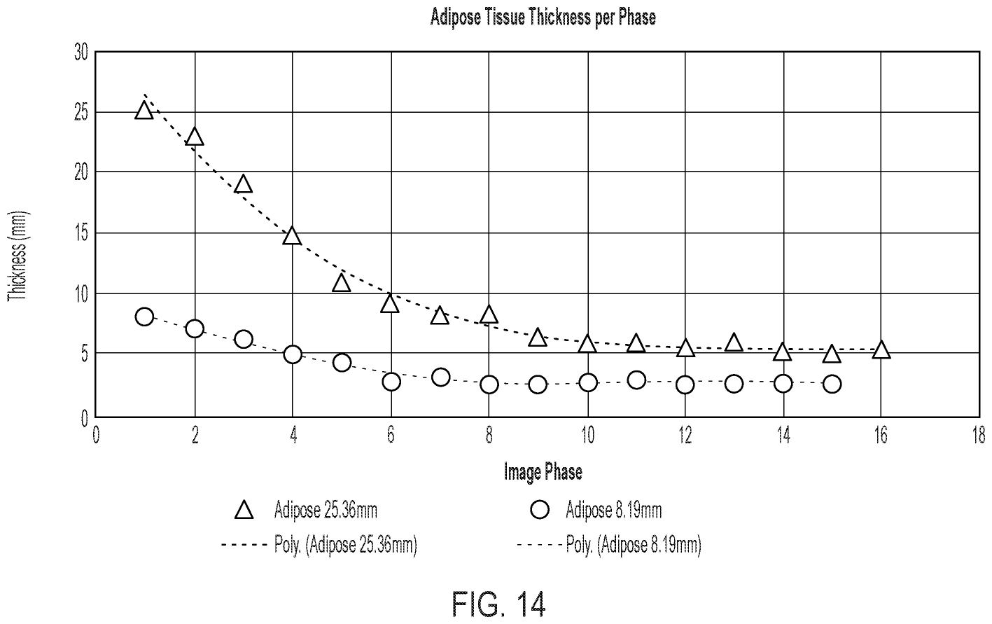

FIG. 14 provides compressive properties of abdominal wall adipose tissue measured using device and MR imaging. For thicker adipose layer the ratio of compression is greater compared to narrower layer. Both layers reach an asymptote at different thicknesses indicating that the applied force is transferred to the muscle at different levels depending on adipose layer thickness. The point at which total applied force is transferred to the muscle layer is important for determining abdominal properties.

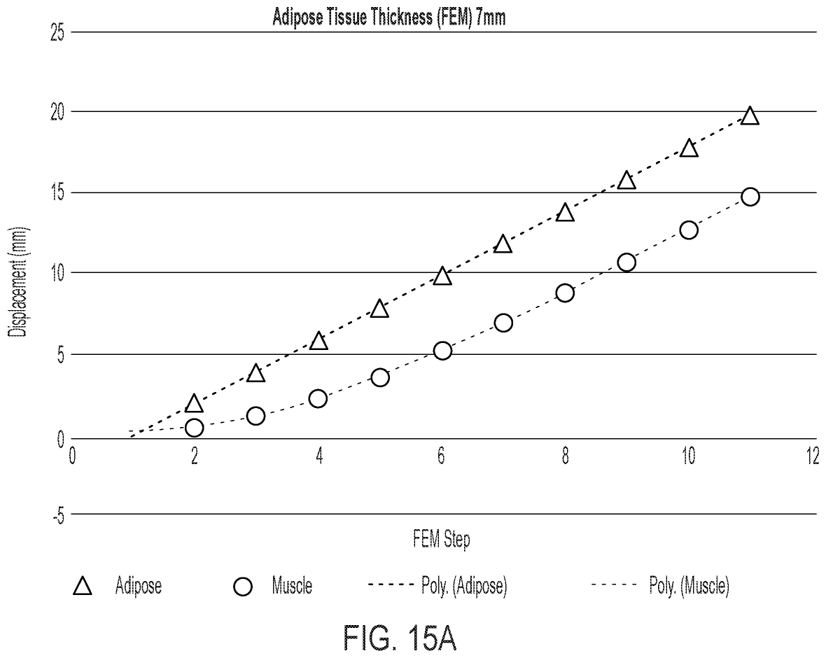

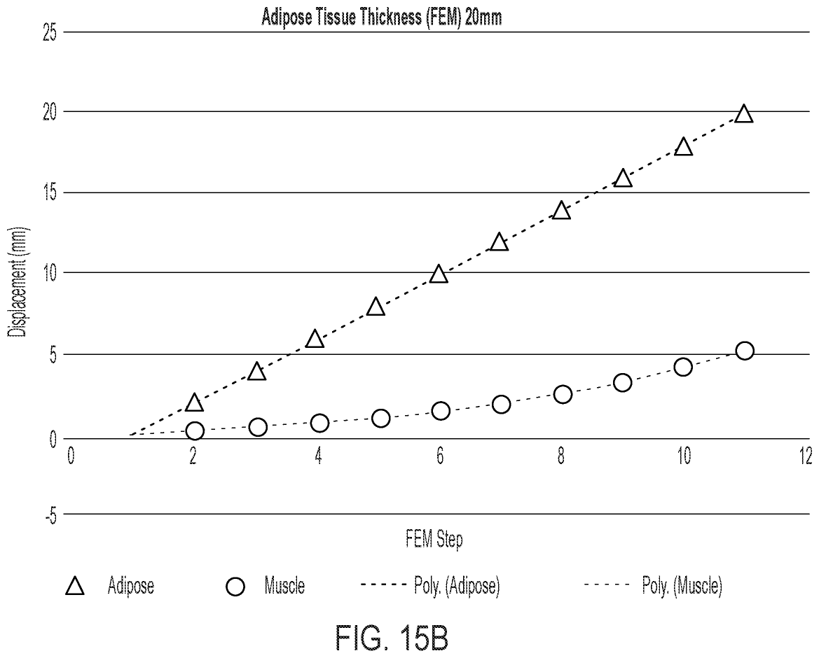

FIGS. 15A and 15B provide example results from FEA modeling showing the effect of adipose tissue thickness on muscle displacement. The simulation correlates well with the observed hernia device and MR imaging results.

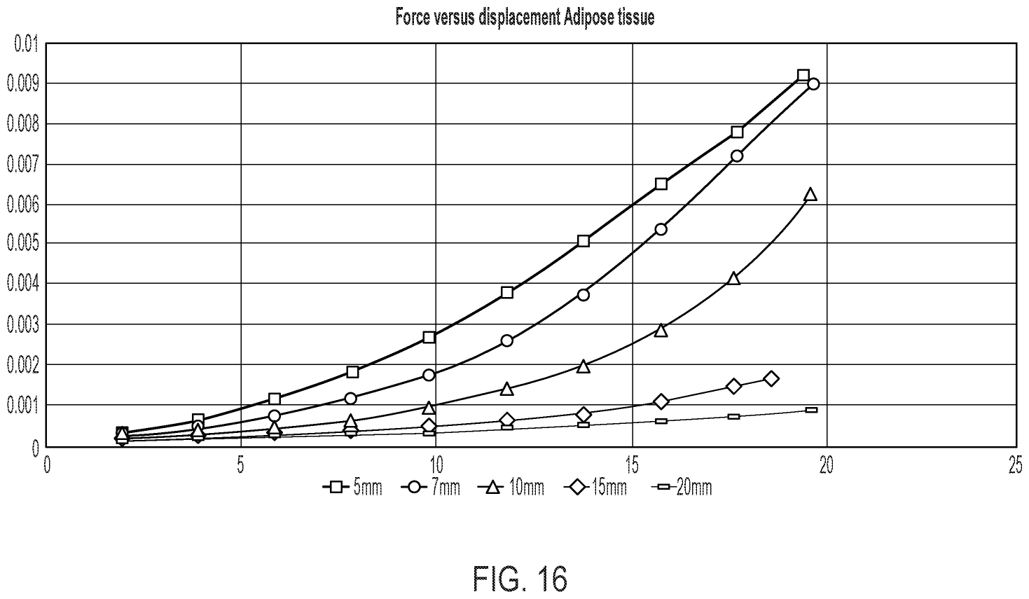

FIG. 16 provides applied force versus adipose tissue displacement generated from FEA. Adipose tissue thickness significantly alters the measured force for the same displacement.

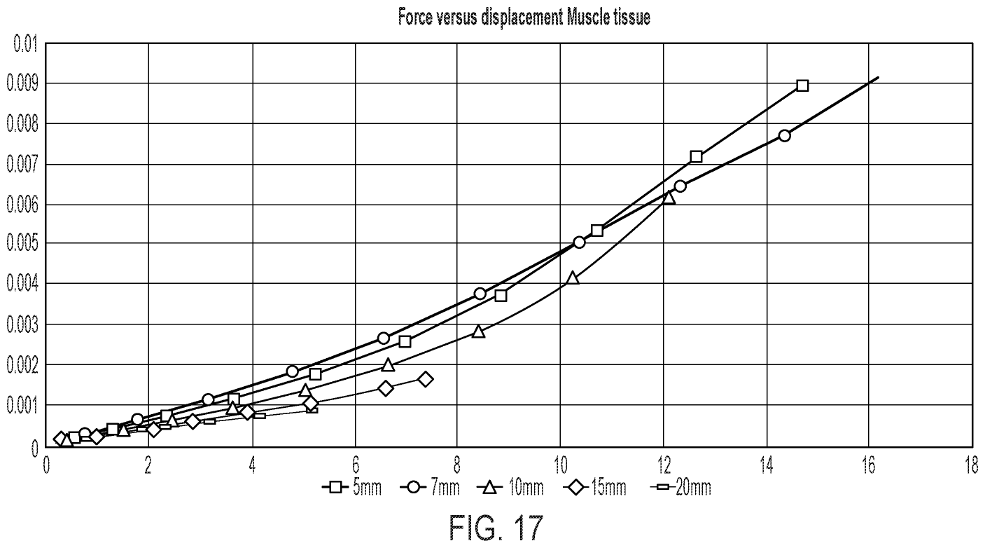

FIG. 17 provides applied force versus muscle tissue displacement generated from FEA. Adipose tissue thickness significantly alters the measured force and displacement of the underlying muscle layer.

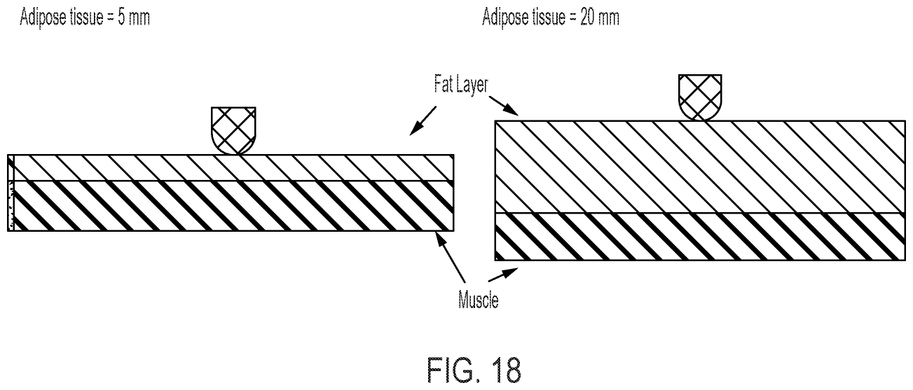

FIG. 18 provides an example FEA model of the abdominal wall and adipose tissue layer. Abdominal wall is modeled as beam supported on two opposing sides with a concentrated force applied at center; a model with adipose tissue of 5 mm is on the left and a model with adipose tissue of 20 mm is on the right. This configuration is analogous to the flexural testing method to measure the stiffness of material under a concentrated load. The adipose tissue is modeled as a material loosely connected to the underlying muscle.

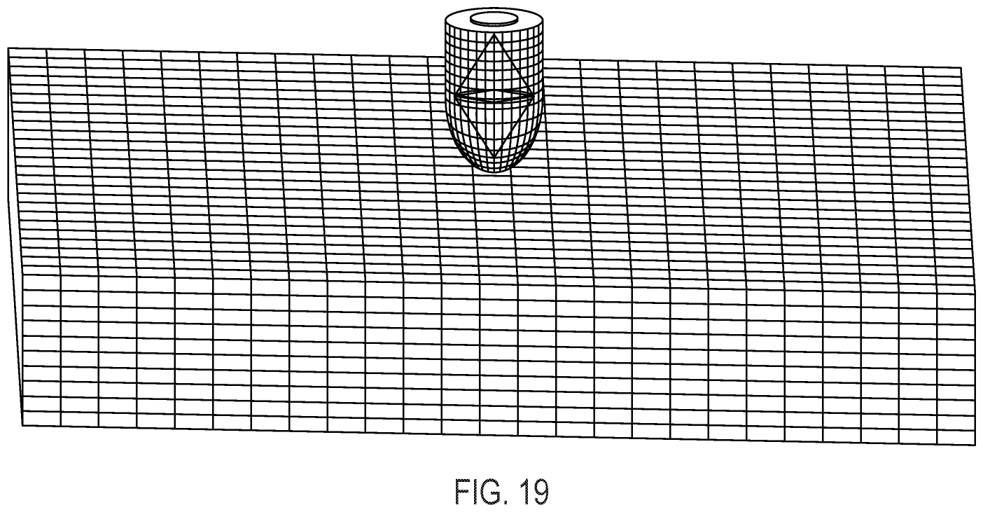

FIG. 19 provides an illustration of a finite element model of abdominal wall and device. The abdominal wall is modeled as a supported beam with two opposite sides fixed. Force is applied at center of beam using an object with a semi-spherical contact point to represent device.

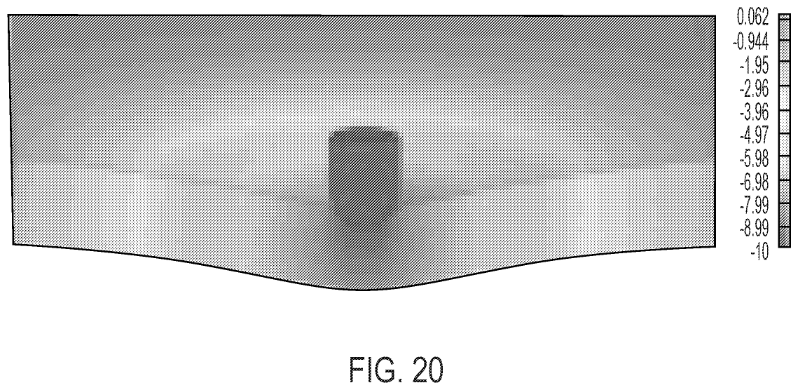

FIG. 20 provides an exemplary view of abdominal wall and device model displacement, highlighting abdominal wall displacement in the z-direction for an applied device force.

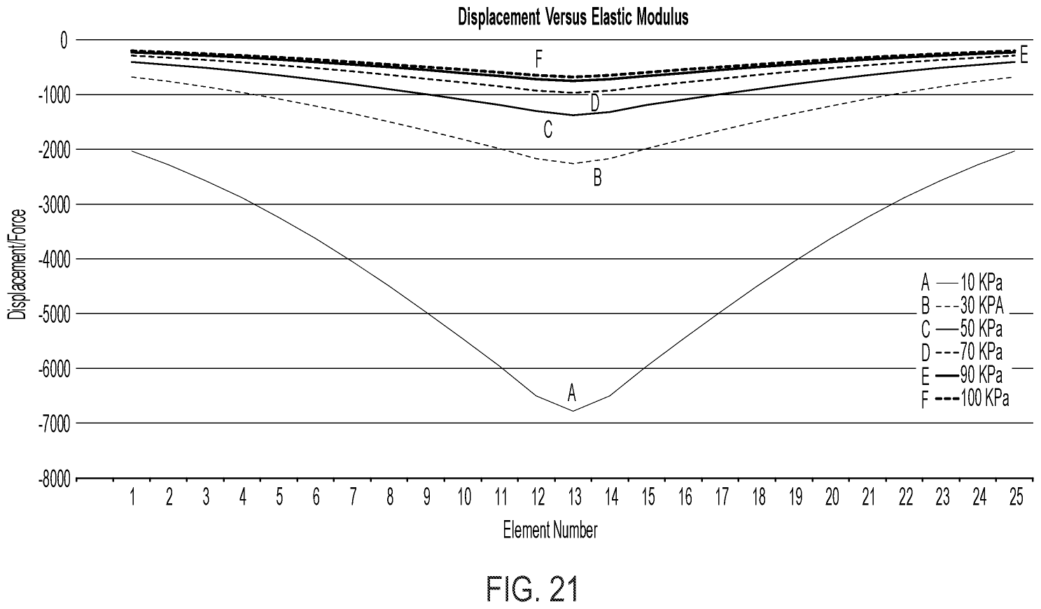

FIG. 21 provides a graph of abdominal wall and device model z-displacement versus elastic modulus.

### EXEMPLARY EMBODIMENTS

The following embodiments are exemplary only and do not limit the scope of the present disclosure or the appended claims.

### Embodiment 1

A method, comprising: with a force applicator, deforming a region of a tissue synchronous with at least one magnetic resonance (MR) imaging sequence that comprises MR tissue tagging.

The tissue can be deformed with a system or device according to the present disclosure. As an example, the tissue can be deformed by a piston or other force applicator synchronous with a MR imaging sequence. The force applicator can include an RF coil, which RF coil can be integrated into the force applicator or attached to the force applicator.

### Embodiment 2

The method of Embodiment 1, further comprising estimating one or more mechanical properties of the tissue. A non-limiting listing of mechanical properties includes, e.g., 2D and/or 3D normal strains, 2D and/or 3D principal strains, stress-strain relationship curves, Young's modulus, flexural modulus, bending modulus, a viscoelastic property (including elastic hysteresis, mechanical creep, mechanical stress relaxation), mechanical frequency response, and/or tissue compressibility.

MRI tissue tracking can be accomplished in a variety of ways. Some exemplary methods include, e.g., SPAMM tagging and DENSE, both of which are known to those of ordinary skill in the art. Example analysis methods include, e.g., HARP, Optical Flow, Snakes, and the like, all of which are known to those of ordinary skill in the art.

### Embodiment 3

The method of any of Embodiments 1-2, wherein the deformation comprises application of a compressive force of about 5 to about 100 Newtons. The compressive force can be, e.g., from about 5 to about 15 Newtons, or even from about 10 to about 15 Newtons. The compressive force can be from, e.g., about 5 to about 100 N, from about 10 to about 90 N, from about 15 to about 80 N, from about 20 to about 70 N, from about 30 to about 60 N, from about 40 to about 50 N, or even about 45 N; the force can also be from about 1 N up to about 20 N or up to about 50 N, from about 2 N to about 19 N, from about 3 N to about 18 N, from about 4 N to about 17 N, from about 5 N to about 16 N, from about 6 N to about 15 N, from about 7 N to about 14 N, from about 8 N to about 13 N, from about 9 N to about 12 N, or even from about 10 N to about 11 N.

One can apply the same force (e.g., 12 N) several times, but one can also apply different forces (e.g., application of 10 N followed by application of 15 N) to a subject during the course of an analysis. The force can thus be a constant force, but can also be varied over time, distance, or both. For example, the force can be 15 Newtons for the first 1 cm of compression, and then be 10 Newtons for the following 2 cm of compression. Different forces can be applied during use of the disclosed technology; as an example a force of 5 Newtons can be applied during a first application of force and a force of 10 Newtons can be applied during a second application of force.

### Embodiment 4

The method of Embodiment 3, wherein the compressive force is effected by movement of a piston, by inflation of a chamber, or both. A piston can contact the tissue directly. A piston can also actuate a membrane or other barrier between the piston and the tissue, as well.

### Embodiment 5

The method of Embodiment 4, wherein the compressive force is effected by movement of a piston.

### Embodiment 6

The method of any of Embodiments 1-5, wherein the region of the tissue is displaced by less than about 10 cm. The region of the tissue can be displaced by, e.g., less than about 9 cm, less than about 8 cm, less than about 7 cm, less than about 6 cm, less than about 5 cm, less than about 4 cm, less than about 3 cm, less than about 2 cm, or even less than about 1 cm. The region of tissue can be displaced by from about 1 to about 10 cm, from about 2 to about 9 cm, from about 3 to about 8 cm, or even from about 4 to about 7 cm.

### Embodiment 7

The method of any of Embodiments 1-6, wherein the region of tissue is displaced in a continuous manner.

### Embodiment 8

The method of any of Embodiments 1-6, wherein the at least one MR imaging sequence has a repetition rate of from about 0.5 to about 4 seconds. A repetition rate of about 2 seconds is considered especially suitable.

### Embodiment 9

The method of any of Embodiments 1-8, further comprising acquiring images, the images being acquired in a continuous manner during active displacement.

### Embodiment 10

The method of any of Embodiments 1-9, further comprising maintaining the force application in a position relative to the tissue and monitoring displacement with a series of time-resolved MR images showing the state of the tissue. This can be accomplished by, e.g., maintaining the tissue in a stretched (or compressed) state and monitoring the tissue's displacement over time.

### Embodiment 11

The method of any of Embodiments 1-10, further comprising reducing the deformation of the tissue and monitoring with the at least one MR imaging sequence a state of the tissue.

### Embodiment 12

A method, comprising: deforming a region of a tissue and while the tissue is at a deformed state and with at least one MR imaging sequence that comprises MR tissue tagging, monitoring displacement at a plurality of time points so as to estimate at least one characteristic of the tissue. Monitoring can also be accomplished by monitoring the tissue at the start of (or before) the deforming and at the completion of the deformation.

### Embodiment 13

A system for magnetic resonance (MR) imaging of an object, comprising: a force applicator configured to effect exertion of a force on the object so as to deform the object; the system further comprising a radio frequency (RF) coil, the RF coil being optionally configured as receive only or as a transmit and receive coil, the RF coil being optionally being configured as a single loop or as a multi-coil array. The force applicator can be configured to effect exertion of the force on the object at least partially synchronous with at least one magnetic resonance (MR) imaging sequence (such as one that comprises MR tissue tagging). An MR imaging sequence can be one wherein images are taken at intervals (e.g., following deformation of tissue) or continuously.

The force applicator can be configured to apply force continuously or at intervals. The system can further comprise a signal source configured to give rise to a signal that allows for measurement of motion of the force applicator; the system optionally comprising an MR imaging sequence using tissue tagging to measure 2D and/or 3D tissue displacements.

A signal source can be used to allow for measurement of the force applicator's velocity and acceleration. Example signal sources include, e.g., a fluid-filled ring or bead that is attached to (or is a part of) a piston rod of the force applicator. The signal source can also be another source of signal that is attached to or incorporated into the force applicator.

### Embodiment 14

The system of Embodiment 13, wherein the force applicator comprises a piston.

### Embodiment 15

The system of Embodiment 14, further comprising two or more different surfaces for patient contact. The surfaces can include varied geometries and surface areas that are actuated by the piston.

### Embodiment 16

The system of Embodiment 13, wherein the piston actuates a contacting cap.

### Embodiment 17

The system of Embodiment 16, wherein the piston defines a cross-sectional dimension, and wherein the contacting cap defines a surface having a cross-sectional dimension that is greater than the cross-sectional dimension of the piston. This can be in the form of a flared cap that flares outward away from an end of the piston that advances the cap. Such a flared cap can be used to compress the fat layer that lies atop a patient's musculature (e.g., their abdominal musculature). After the fat layer is compressed, the compressive force from the force applicator is transferred more directly to the patient's abdominal musculature through the compressed fat layer.

### Embodiment 18

The system of Embodiment 16, wherein the piston defines a cross-sectional dimension, and wherein the contacting cap defines a surface having a cross-sectional dimension that is less than the cross-sectional dimension of the piston. This can be in the form of a tapered or pointed cap that tapers away from the end of a piston that advances the cap. A tapered cap can act to part the fat layer that lies atop a patient's musculature, thus affording more direct access to the patient's musculature.

### Embodiment 19

The system of any of Embodiments 13-18, further comprising a holder portion configured to receive the force applicator. A holder portion can be, e.g., a socket, a cup, a collar, a ring, an extension, a protrusion, a rim or lip, or other feature that receives the force applicator and can maintain the force applicator in position. As one example, a holder portion can be a collar into which the force applicator (e.g., a piston) engages. The holder portion and the force applicator can comprise one or more complementary features (e.g., a slot/tab pairing) that facilitate engagement between the holder portion and force applicator.

### Embodiment 20

The system of Embodiment 19, wherein the holder portion comprises a socket.

### Embodiment 21

The system of Embodiment 20, wherein the holder portion is configured to releasably secure the force applicator. As an example, the holder portion and force applicator can comprise a bayonet-type coupling.

### Embodiment 22

The system of Embodiment 19, further comprising one or more spacers positioned in the holder portion so as to maintain a position of the force applicator in a receiver portion. The one or more spacers can act to suspend the force applicator at a certain position so as to control the amount by which the force applicator can extend relative to the patient. For example, a piston-type force applicator can be received by a holder portion such that the piston can extend by a maximum of 7 cm beyond the holder portion, thus deforming a patient's tissue by that 7 cm. If a spacer having a height of 1.5 cm were placed into the holder portion and then the piston-type force applicator were placed atop the spacer, the piston could then extend by 7 cm-1.5 cm=5.5 cm. In this way, a user can add (or remove) spacers so as to achieve a level of patient comfort while also achieving a level of diagnostic information.

### Embodiment 23

The system of Embodiment 13, wherein the force applicator comprises a pneumatic chamber.

### Embodiment 24

The system of any of Embodiments 13-23, further comprising a magnetic field source.

### Embodiment 25

The system of Embodiment 24, wherein the system is configured to effect exertion of the force on the object synchronous with a cycle of MR signal acquisition

### Embodiment 26

The system of Embodiment 23, wherein the system is configured to collect a signal from the RF coil during MR sequence acquisition.

### Embodiment 27

The system of Embodiment 26, the system being further configured to estimate a mechanical property of the object based in part on an RF coil signal.

### Embodiment 28

The system of Embodiment 27, wherein the mechanical property is 2D and/or 3D normal strains, 2D and/or 3D principal strains, stress-strain relationship curves, Young's modulus, viscoelastic properties (including elastic hysteresis, mechanical creep, mechanical stress relaxation), mechanical frequency response, and/or tissue compressibility.

### Embodiment 29

The system of any of Embodiments 13-28, further comprising one or more components configured to maintain the force applicator in a position relative to the object. Such components can be, e.g., belts, straps, elastic bands, and the like.

### Embodiment 30

The system of any of Embodiments 12-29, wherein the system is configured for use on a human patient.

### Embodiment 31

A system, comprising: a force applicator configured to apply a compressive force to a subject's tissue synchronous with a cycle during MR sequence acquisition, a radio frequency (RF) coil; the RF coil being configured as a receive only or a transmit and receive coil, the RF coil being configured as a single loop or a multi-coil array, and an MR imaging sequence to generate images synchronous with tissue displacement.

### Embodiment 32

The system of Embodiment 31, further comprising a processor configured to estimate one or more mechanical properties of the subject's tissue based at least in part on the tissue signals.

### Embodiment 33

A method, comprising: synchronous with at least one magnetic resonance (MR) sequence that comprises MR tissue tagging, applying a force to a location on a subject so as to displace adipose tissue of the subject; collecting at least one signal related to the application of the force; and relating at least one signal to an estimated mechanical property of the adipose tissue. As described elsewhere herein, the force can be applied by a force applicator that is MR-compatible.

### Embodiment 34

The method of claim 33, wherein the adipose tissue is located in the subject's abdomen.

### Embodiment 35

The method of claim 33, wherein relating at least one signal to an estimated mechanical property of the adipose tissue comprises relating the force to a displacement of the adipose tissue. The relating can be, e.g., in the form of an FE model.

### Embodiment 36

The method of claim 33, wherein that at least one signal is indicative of a displacement of the adipose tissue.

### Embodiment 37

The method of any one of claims 33-36, wherein the mechanical property of the adipose tissue is at least one of a flexural modulus, an elastic modulus or a tissue displacement velocity.

### Embodiment 38

The method of any one of claims 33-37, further comprising at least partially releasing the force.

### Embodiment 39

The method of claim 38, further comprising collecting at least one signal related to the releasing of the force. As an example, one can apply and release a force over one cycle or even over a plurality of cycles, thereby allowing for the collection of multiple data points so as to allow the user to determine an average value, a mean value, or other figure of merit.

### Embodiment 40

The method of any one of claims 33-39, further comprising applying the force so as to displace muscle tissue that underlies the adipose tissue, further comprising optionally locating a boundary between the adipose tissue and the muscle tissue based on a change in the signal.

### Embodiment 41

The method of claim 40, further comprising relating the at least one signal to an estimated mechanical property of the muscle tissue.

### Embodiment 42

The method of claim 41, wherein the signal is indicative of a displacement of the muscle tissue.

### Embodiment 43

The method of claim 41, wherein the estimated mechanical property is at least one of a bending modulus, elastic modulus, or a tissue velocity.

### Embodiment 44

The method of any one of claims 40-43, further comprising at least partially releasing the force.

### Embodiment 45

The method of claim 44, further comprising collecting at least one signal related to the releasing of the force.

### Embodiment 46

The method of claim 41 or claim 45, further comprising relating the estimated mechanical property of the muscle tissue to the presence of a condition in the muscle tissue.

### Embodiment 47

The method of claim 46, wherein the condition is an altered stiffness, the altered stiffness optionally being indicative of herniation.

### Embodiment 48

The method of any one of claims 33-47, wherein the applying is effected by a force applicator.

### Embodiment 49

The method of claim 48, further comprising relating a displacement of the force applicator to an estimated mechanical property of the muscle tissue.

### Embodiment 50

The method of any one of claims 33-49, further comprising performing image processing so as to distinguish between the adipose tissue and muscle tissue.

### Embodiment 51

The method of any one of claims 33-50, further comprising applying a force to a second location on a subject so as to displace adipose tissue of the subject; collecting at least one signal related to the application of said force; and relating the at least one signal to an estimated mechanical property of the adipose tissue.

As described elsewhere herein, one can compare the estimated structural property (e.g., a stiffness, a flexural modulus, a tissue displacement velocity, an elastic modulus) of a subject to a corresponding property so as to diagnose a subject's condition or predict a subject's condition. For example, if one or more of the flexural modulus, tissue displacement velocity, and/or elastic modulus of a subject's muscle tissue is near or even below a particular threshold value, that patient can be categorized into a particular risk group. For example, a patient having an abnormally low flexural modulus of abdominal muscle tissue can be treated differently (e.g., with a different surgical technique) than a patient with a normal-range flexural modulus.

### Embodiment 52

A system, the system comprising: a MR-compatible force applicator configured to apply a force to a location on a subject synchronous with at least one magnetic resonance (MR) sequence that comprises MR tissue tagging, the force being applied so as to displace adipose tissue of the subject; and a processor, the processor configured to relate a signal related to the application of the force to an estimated mechanical property of the adipose tissue.

### Embodiment 53

The system of claim 52, the processor being configured to relate the signal to an estimated property of muscle tissue of the subject.

### Embodiment 54

The system of any one of claims 52-53, wherein the estimated property is at least one of a bending modulus, an elastic modulus or a tissue displacement velocity.

### Embodiment 55

The system of any one of claims 52-54, wherein the system comprises a plurality of MR-compatible force applicators. As an example, a system can include two, three, four, or even five force applicators. This in turn allows a system to determine muscle and/or adipose tissue mechanical properties at a plurality of locations of a subject's abdomen. This can in turn provide information concerning optimal (and/or sub-optimal) sites for surgical procedures, and can also help to guide surgical procedure strategy such that a surgical procedure is performed at a location that is most likely to best tolerate the procedure and/or experience relatively fewer complications from the procedure.

Although MR-compatible force applicators can be used, this is not a requirement for all embodiments. As an example, a system can also include a force applicator (whether MR-compatible or not) configured to apply a force to a location on a subject, the force being applied so as to displace adipose tissue of the subject; and a processor, the processor configured to relate a signal or other information related to the application of the force to an estimated mechanical property of the adipose tissue. Such a system can be configured to operate without imaging the adipose tissue (or muscle tissue adjacent to the adipose tissue).

As another example, a system can also include a force applicator (whether MR-compatible or not) that configured to apply a force to a location on a subject, the force being applied so as to displace adipose tissue of the subject; and a processor, the processor configured to relate a signal or other information related to the application of the force to an estimated mechanical property of muscle tissue adjacent to the adipose tissue. Such a system can be configured to operate without imaging the adipose tissue (or muscle tissue adjacent to the adipose tissue).

### Embodiment 56

A method, comprising: relating a displacement velocity, a bending modulus, or an elastic modulus of the tissue to a physiological condition of the tissue following application of a force to a region of a subject.

### Embodiment 57

The method of claim 56, wherein the physiological condition is an altered stiffness, the altered stiffness optionally being indicative of herniation.

### Embodiment 58

The method of claim 56, wherein the velocity is determined by relating an MR signal to the displacement.

### Embodiment 59

The method of claim 56, wherein the bending modulus or the elastic modulus is determined by relating an MR signal to the force.

### Embodiment 60

The method of claim 56, wherein the tissue is a muscle tissue.

### Embodiment 61

The method of claim 56, wherein the tissue is an adipose tissue.

### Embodiment 62

The method of claim 60, further comprising distinguishing the muscle tissue from an adipose tissue that overlies the muscle tissue.

### Embodiment 63

A method, comprising: with a force applicator that travels a distance, applying a force to a subject (e.g, to their abdomen); relating one or both of the force and the distance to an estimated mechanical property of a muscle tissue of the subject; the relating being at least partially based on an estimated mechanical property of an adipose tissue.

### Embodiment 64

The method of claim 63, wherein the estimated mechanical property of the adipose tissue is based on a sampling of two or more adipose tissue thicknesses.

Without being bound to any particular theory or embodiment, one can, for example, use calipers or other instrumentation (including non-imaging instruments) to measure adipose tissue thickness. One can then use that measurement in combination with a non-imaging device and FEM to determine, e.g., abdominal wall stiffness. In this way, one can estimate the mechanical property of muscle tissue without also imaging that muscle tissue.

As an example, one can—as described elsewhere herein—construct a library of adipose tissue mechanical properties, e.g., as a function of the thickness of the adipose tissue. In this way, when one then measures the actual adipose tissue thickness of a subject (e.g., using a caliper and obtaining a adipose tissue thickness of 3 cm), one can then relate that 3 cm measurement to the expected mechanical properties of adipose tissue having a thickness of 3 cm, based on the information in the adipose tissue library. One can then use a non-imaging force applicator that then travels a distance (e.g., a plunger or other device, such as those described elsewhere herein) to apply a force to the subject and then, using the expected mechanical properties of the subject's 3 cm layer adipose tissue, isolate the mechanical properties of the subject's muscle tissue from the force-distance information collected by the force applicator. In this way, a user armed with a library of adipose tissue properties (e.g., as a function of thickness) can in turn estimate a mechanical property of a subject's muscle tissue without necessarily imaging that muscle tissue. (As described elsewhere herein, a user can also image the subject's muscle tissue.)

### Embodiment 65

A method, comprising: with a force applicator that travels a distance, applying a force to a subject (e.g, to their abdomen, to their limb, or to another location of the subject); relating one or both of the force and the distance to an estimated mechanical property of a muscle tissue of the subject, the relating being accomplished without imaging the muscle tissue.

In this way, one can estimate a mechanical property of a subject's muscle tissue without also having to image that tissue. As one example, one can monitor the force and/or displacement of a force applicator that applies a force to a patient's abdomen. The user can then determine the stage during force application at which the muscle tissue deforms (which can, in some instances, involve determining the stage at which adipose tissue disposed adjacent to the muscle tissue has itself deformed to the point that deformation of the muscle tissue begins), and from that force-displacement information, relate that information to the mechanical property (e.g., flexural modulus) of the muscle tissue.

### Embodiment 66

The method of claim 65, further comprising measuring the distance by exterior observation of the force applicator.

### Embodiment 67

The method of any one of claims 65-66, further comprising determining a distance traveled by the force applicator, upon which distance the force applicator deforms the muscle tissue.

### Embodiment 68

The method of any one of claims 65-67, the relating being at least partially based on an estimated mechanical property of an adipose tissue of the subject. As described elsewhere herein, the estimated mechanical property of an adipose tissue of the subject can be based on estimated (or measured) mechanical properties of adipose tissues of other subjects, e.g., properties from a library of adipose tissue properties.

### Example Results

IRB approval was obtained prior to the start of the study and was HIPAA compliant. A custom device was built that deforms the abdominal wall muscle synchronous with the imaging cycle. Deformation was performed using an external device consisting of an MR compatible pneumatically actuated air cylinder (IPS, Irvine, Calif.) that was mounted to an RF coil. Pneumatic valves triggered by a timing board controlled the rates of extension and retraction of the air cylinder piston-rod. Maximum extension of the piston rod was 5 cm.

The timing board also triggered the imaging sequence, which was a 2D SPGR gated multiphase acquisition with tissue tagging. Scan parameters were: TE=1.75 ms, TR=3.9 ms, FOV=320×210 mm, 4 mm slice thickness, 6 mm tag spacing, using 5 views per cardiac segment. The sequence was gated to the air cylinder cycle time, which was 1800 ms. The scan was performed in a breath-hold which was 27 seconds.

Five imaging studies were successfully performed on normal volunteers. The air cylinder was able to produce sufficient deformation of the abdominal wall muscle synchronous with the tissue tagged imaging sequence. Images from a volunteer study are shown in FIG. 2. Muscle deformation is clearly seen in the final frames.

MR tissue tagging with synchronous displacement of the abdominal wall was shown to provide measurements of muscle deformation. This allows analysis of the mechanical properties of muscle for noninvasive pre-operative prediction of successful fascial closure in hernia repair.

Further Disclosure

In one embodiment of the disclosed technology, MR tagged images (e.g., SPAMM) are created with an embedded grid of altered signal that deforms as the body tissue moves. The grid acts as a fiducial landmark, adding confidence to tracking of motion, particularly in areas of isointense signal. The MR tagged abdominal images can be analyzed using a variety of methods, e.g., an optical flow method (OFM) that tracks every pixel in the image, using both the grid contrast as well as local tissue texture for uniquely identifying pixels. From these displacement fields, the viscoelastic properties are subsequently derived. With image resolution of, e.g., between 0.75 and 1.5 mm (1 mm is a suitable value), the regional tissue properties are reported with the same high-resolution. This approach can be contrasted with tracking so-called tag intersections, which are spaced from 4-6 mm and have significantly reduced spatial resolution in pixel tracking and subsequent assessment of local tissue properties.

The disclosed technology also tracks muscle displacement at a comparatively high temporal resolution. Imaging occurs during the application of the force, and image frames can be acquired with, e.g., 20-50 ms resolution. Continuous imaging allows robust estimates of total displacement and also allows tracking of the extent deformation and also deformation path. This can be contrasted with other approaches that image tissue before and after force application but do not image tissue during force application.

Example Modeling Disclosure

Abdominal Wall Finite Element Model

One can use an abdominal wall FE model is to simulate the displacement and deformation obtained in-vivo with the ultimate goal to estimate axial and transverse elasticity. Since the layers of the abdominal wall in-vivo do not function in isolation but rather as an integrated system, the model treats the wall as a composite structure where multiple muscles and intra-abdominal pressure are combined into a total elasticity (FIG. 19). FE model represents abdominal wall as a 3D plate where displacements and deformation are analyzed for two orthogonal planes intersecting the model geometry at the center of the load representing an axial and transverse beam respectively. The initial model uses a simplified 3D geometry where the thickness of the wall is set at 10% of the muscle length in both x and y directions. One can also use segmented 3D MRI images of the abdominal muscles and/or adipose tissue layer to generate the geometry for the model. Actual wall geometry will enhance the accuracy of the model improving correlation between model generated results and in-vivo measurements. Meshing the more complex geometry will involve exploring the use of different element types to determine which provide the more accurate results.

Loading and Boundary Conditions

Model loading simulates the application of force/displacement in-vivo. Load is applied using an object that represents the semi-spherical contact geometry of the in-vivo device (FIG. 19). In the model, load is defined as displacement in the z-direction at the center element of the abdominal wall geometry. The applied load is measured as the pressure on the contacting element which corresponds to the force measured in-vivo. Model boundary conditions are applied to both the load object and abdominal wall geometry. The load object can be defined to be rigid and incompressible ensuring all applied force is transmitted directly to wall geometry as opposed to deforming load object. In addition, force can be applied only to the contact surfaces between the load object and wall geometry. To simulate the in-vivo muscle attachment to rigid boney structures two opposing sides of the wall geometry are restricted in x, y, and z displacement while, the remaining two sides are unrestricted.

Material Properties

A finite element (FE) model uses elastic isotropic material properties for the abdominal wall. This assumes the properties of the material are directionally independent where the stress (σ)/strain (ε) equations (eq1) are constructed from only two elastic constants: elastic modulus (E) and Poisson's ratio (v).

εₓₓ=[σₓₓ−v(σ_{yy}+σ_{zz})]/E

ε_{yy}=[σ_{yy}−v(σ_{zz}+σₓₓ)]/E

ε_{zz}=[σ_{zz}−v(σₓₓ+σ_{y})]/E (Eq. 1)

Use of this material type can be valid for a composite structure where transverse properties vary across the layers, creating a pseudo-isotropic material. One can also consider the use of transversely isotropic material properties to represent the abdominal wall where the material is isotropic in one plane but has a different stiffness in an orthogonal plane (Eq. 2). In this model, the stiffer plane represents the pseudo-muscle fiber orientation.

$${ɛ}_{1} = \frac{{σ}_{1}}{\text{?}} - \frac{\text{?}}{\text{?}} ( {σ}_{2} - {σ}_{3} ) + \text{?} Δ T \\ {ɛ}_{2} = - \frac{\text{?}}{\text{?}} \text{?} + \frac{{σ}_{2}}{\text{?}} - \frac{\text{?}}{\text{?}} {σ}_{3} + \text{?} Δ T \\ \text{?} = - \frac{\text{?}}{\text{?}} {σ}_{1} - \frac{\text{?}}{\text{?}} {σ}_{2} + \frac{{σ}_{3}}{\text{?}} + \text{?} Δ T \\ \text{?} \text{indicates text missing or illegible when filed} \qquad ( Eq . 2 )$$

Numerical Analysis

In an example analysis, an abdominal wall FE model is created using modeling software and analyzed using a nonlinear finite element solver which generates 3D visualization and animation of finite element models (FIG. 20). From the 3D visualization, displacements, strains, and stresses for each element can be analyzed and displayed such as the z-displacement normalized by force for varying elastic moduli shown in FIG. 21.

To determine the abdominal wall material parameters, optimization software can be used. The desired outcome is the optimal set of parameters that minimize the mean squared error (MSE) between the FE predicted strain and the in-vivo strain/displacement measured from tissue tagged MRI. Strain/displacement is compared at roughly 20 locations within the abdominal wall. The parameters to be optimized are the abdominal wall elastic modulus (E_{A}-axial elastic modulus, E_{T}-transverse elastic modulus). The MSE is given by Eq. 3:

$$MSE = {\sum }_{n = 1}^{N} {\sum }_{{i = 1 , 2 , 3 ;}_{j = 1 , 2 , 3 ;}}^{ } {{w}_{n} ( {E}_{ij , n} - {\overline{E}}_{ij , n} )}^{2} \qquad Eq . 3$$

where N is the total number of strain/displacement points, E_{ij,n} the predicted FE strain and E_{ij,n} the measured strain for each point, and wₙ a weighting factor.

What is claimed:

1. A method, comprising
with a force applicator, deforming a region of a tissue synchronous with at least one magnetic resonance (MR) imaging sequence that comprises MR tissue tagging.

2. The method of claim 1, further comprising estimating one or more mechanical properties of the tissue.

3. The method of claim 1, wherein the deformation comprises application of a compressive force of about 5 to about 100 Newtons.

4. The method of claim 3, wherein the compressive force is effected by movement of a piston, by inflation of a chamber, or both.

5. The method of claim 1, further comprising acquiring images wherein the images are acquired in a continuous manner during active displacement.

6. A system for magnetic resonance (MR) imaging of an object, comprising;
a force applicator configured to effect exertion of a force on the object so as to deform the object;
the system further comprising a radio frequency (RF) coil,
the RF coil being optionally configured as receive only or transmit and receive coil,
the RF coil being optionally being configured as a single loop or a multi-coil array.
the system further comprising a signal source configured to give rise to a signal that allows for measurement of motion of the force applicator;
the system optionally comprising an MR imaging sequence using tissue tagging to measure 2D and/or 3D tissue displacements.

7. The system of claim 6, further comprising a holder portion configured to receive the force applicator.

8. The system of claim 7, wherein the holder portion comprises a socket.

9. The system of claim 7, wherein the holder portion is configured to releasably secure the actuator.

10. The system of claim 7, wherein the system is configured to effect exertion of the force on the object synchronous with a cycle of MR signal acquisition.

11. The system of claim 7, the system being further configured to estimate a mechanical property of the object based in part on the signal.

12. The system of claim 11, wherein the mechanical property is 2D and/or 3D normal strains, 2D and/or 3D principal strains, stress-strain relationship curves, Young's modulus, flexural modulus, bending modulus, a viscoelastic property, mechanical frequency response, tissue compressibility, or any combination thereof.

13. A method, comprising: relating a displacement velocity, a bending modulus, or an elastic modulus of the tissue to a physiological condition of the tissue following application of a force to a region of a subject.

14. The method of claim 13, wherein the physiological condition is an altered stiffness, the altered stiffness optionally being indicative of herniation.

15. The method of claim 13, wherein the velocity is determined by relating an MR signal to the displacement.

16. The method of claim 13, wherein the tissue is a muscle tissue.

17. The method of claim 16, further comprising distinguishing the muscle tissue from an adipose tissue that overlies the muscle tissue.

18. A method, comprising:
with a force applicator that travels a distance, applying a force to a subject's abdomen;
relating one or both of the force and the distance to an estimated mechanical property of a muscle tissue of the subject;
the relating being at least partially based on an estimated mechanical property of an adipose tissue.

19. The method of claim 18, wherein the estimated mechanical property of the adipose tissue is based on a sampling of two or more adipose tissue thicknesses.

20. A system, comprising:
a force applicator configured to apply a force to a location on a subject,
the force being applied so as to displace adipose tissue of the subject; and
a processor, the processor configured to relate a signal or other information related to the application of the force to an estimated mechanical property of muscle tissue adjacent to the adipose tissue.
